(19) United States
(12) Patent Application Publication
(10) Pub. No.: US 2024/0238302 A1
(43) Pub. Date: Jul. 18, 2024

(54) KIT FOR MIGRAINE MANAGEMENT

(71) Applicant: CHRISTOPHER MILLER, ALAMEDA, CA (US)

(72) Inventor: CHRISTOPHER MILLER, ALAMEDA, CA (US)

(21) Appl. No.: 18/096,471

(22) Filed: Jan. 12, 2023

(51) Int. Cl. A61K 31/522; A61J 7/00; A61K 9/00; A61K 9/06; A61K 9/20; A61K 31/05; A61K 31/122; A61K 31/198; A61K 31/525; A61K 33/06; A61K 36/185; A61K 36/28; A61K 36/53; A61K 36/534; A61K 36/81; A61K 36/9068

(52) U.S. Cl. CPC A61K 31/522; A61J 7/0084; A61K 9/0014; A61K 9/006; A61K 9/06; A61K 9/20; A61K 31/05; A61K 31/122; A61K 31/198; A61K 31/525; A61K 33/06; A61K 36/185; A61K 36/28; A61K 36/53; A61K 36/534; A61K 36/81; A61K 36/9068

(57) ABSTRACT

The invention provides a kit for migraine management. More specifically at least three pharmaceutical compounds are provided which seek, in combination, to provide relief to a migraine sufferer in the instance of a present migraine event.

### FIELD OF THE INVENTION

The invention provides a kit for migraine management. More specifically at least three pharmaceutical compounds are provided which seek, in combination, to provide relief to a migraine sufferer in the instance of a present migraine event.

### BACKGROUND OF THE INVENTION

Migraine is an age old disease which has been described and dealt with in various ways throughout history by many cultures and civilization. For example, old English literature described as “Hemicrania’, implying that migraine headache is unilateral in the head. However, migraine does not always attack in a unilateral manner. It took Several centuries to understand the Scientific basis and recognize the wide clinical Spectrum of this very common illness.

As of recent times, in the United States alone, there are 28 million people Suffering migraine. Of that population, approximately 21 million are female and 7 million are male. One in four households has at least one migraine prevalence peaks in the 25 to 55 year age ranges in both genders. In the 1999 HIS (International Headache Society) estimate, 52% of the total population of migraine cases remain undiagnosed. The undiagnosed Sufferers are most likely Self-medicating with over the counter medications. “Migraine Awareness’, i.e., public knowledge of migraine illness, is much more prevalent in the urban areas than in the interior heartland of the United States, for example.

Additionally, migraine is the number one cause of lost work days. Migraine Sufferers visit emergency rooms and doctors' offices more frequently than non-migraine patients. The total cost of lost work in the United States alone is approximately in the range of 5.6 to 17.2 billion dollars a year. Attendant health care costs rival these FIGURES.

Despite tremendous economic pressures to develop a “magic bullet” style treatment useful in treating the full range of the illness, no manufacturer has done SO. For example, a most popular migraine remedy, Sumatriptan, has been used to treat around 140 million cases. However, this drug addresses just one aspect of the complex migraine illness. Subjects who are being treated with Sumatriptan most likely take other drugs, over the counter or prescription, to treat aspects of the disease that Sumatriptan does not address. There remains a need for a more convenient approach to complete migraine treatment wherein all of the components and medications necessary for migraine treatment are provided.

The pathogenesis of migraine may be summarized by: (1) genetic pre-disposition (Almost 90% of migraine Sufferers have a family history of the illness); (2) Environmental factors such as food, atmospheric and climatic changes, emotional upheavals, physical Stress, sleep deprivation, and Social aggravations, and (3) The “cascade phenomenon of migraine”, neurochemical changes occurring in the brain.

The most important chemical change in the brain in migraine is the decline in the level of the neurotransmitter serotonin. All modern treatment methods to treat migraine attacks use a generic group of drugs called Triptans. Triptans work by elevating the brain Serotonin levels through action at the nerve junctions, Synapses. This triggering event results in the release of a variety of other neuropeptides and around the blood vessels in the meninges, i.e., covering of the brain, and in the brain itself. Some of these important neuropeptides are bradykinin, prostaglandin. A few of the important mono-amines are dopamine and norepinephrine. Their roles are described in further detail below.

Migraine is a complex illness in which not only serotonin, but other neuro- are implicated. The acute aseptic, i.e., non-infectious, inflammatory reaction of the meninges which causes the Severe headache is mediated through the release of a chemical, prostaglandin. Thus, the common anti-inflammatory drugs like Aspirin, Tylenol, Ibuprofen, and the like are effective in treating the Severe headache in migraine attacks because these drugs antagonize prostaglandins.

During a migraine attack, there is a release of norepinephrine which accounts for the sense of anxiety, palpitation and high blood pressure in the migraine sufferer. Anxiety relieving drugs like Valium, Xanax and Paxil are often useful in treating migraine.

Release of another important chemical substance called dopamine explains the nausea and vomiting and the tremendous mood Swings in migraine. Reglan (Chlorpropamide) is a dopamine blocking agent and hence is success fully used in the acute stages of migraine to treat accompanying Symptoms other than the headache, such as nausea and mood swings.

The drug manufacturers typically put out one single product in the market and promote it studiously. There are four different Triptan preparations already being marketed and several more are expected soon. However, as mentioned above, a “single drug” approach using Triptan, for example, cannot treat migraine adequately.

Triptans are a family of tryptamine-based drugs used as abortive medication in the treatment of migraines and cluster headaches. This drug class was first commercially introduced in the 1990s. While effective at treating individual headaches, they do not provide preventive treatment and are not considered a cure.

A well experienced migraine doctor skillfully prescribes a combination of drugs to treat acute migraine. Under current State of the art, depending upon the presenting manifestation of a migraine attack, the migraine doctor may prescribe a Triptan, e.g., Imitrex, an anti-prostaglandin drug, e.g., Ibuprofen and an anti-dopamine drug, Reglan, Patients, very often, use this “cocktail” approach on their own. Surprisingly: it is not at all uncommon to find doctors prescribing only Single drug treatment for migraine, which leaves the patient only with partial relief of headache, leaving them unhappy and Seeking other opinions. As described above, to successfully treat a migraine attack, there exists the need to use a combination of several drugs. Yet, no manufacturer has assembled a kit with Such a combination.

### SUMMARY OF THE INVENTION

Accordingly, the invention provides a kit for migraine management, comprising a first container having a first therapeutically effective dosage of an efficacious pharmaceutical compound useful for treating a medical condition; a second container having a second therapeutically effective dosage of an efficacious pharmaceutical compound useful for treating a medical condition; a third container having a third therapeutically effective dosage of an efficacious pharmaceutical compound useful for treating a medical condition; and one or more printed instructions setting forth the safe and effective use of the contents of said kit.

The kit further comprises a computer application (i.e., an “app”) usable on a personal mobile device by a user. Examples of suitable personal mobile devices herein are a cellphone, laptop computer, personal computer, computer pad device from brands like APPLE©, DELL©, SAMSUNG©, and ANDROID© to name but a few.

In practice, The kit for migraine management tracks the occurrence, severity and length of a user's migraine headache. The app may also contain printed instructions viewable on said personal mobile device.

Herein, the first therapeutically effective dosage of an efficacious pharmaceutical compound is administered sublingually, i.e., under the tongue. The second therapeutically effective dosage of an efficacious pharmaceutical compound herein is administered orally for consumption through the user's esophagus and into the user's stomach. The kit for the third therapeutically effective dosage of an efficacious pharmaceutical compound useful for treating a medical condition is topically administered and is best provided in the form of a cream.

### BRIEF DESCRIPTION OF THE FIGURES

The various exemplary embodiments of the present invention, which will become more apparent as the description proceeds, are described in the following detailed description in conjunction with the accompanying drawings, in which:

FIG. 1 is a schematic drawing of the kit herein showing the various components of the kit.

### DETAILED DESCRIPTION OF THE INVENTION

The following description is provided as an enabling teaching of the present systems, and/or methods in its best, currently known aspect. To this end, those skilled in the relevant art will recognize and appreciate that many changes can be made to the various aspects of the present systems, and/or methods described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

As used throughout, the singular forms “a,” “an” and “the” include plural referents unless the context clearly dictates otherwise. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might,” or “may,” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods.

Thus, if there are a variety of additional steps that can be performed it is understood that each of these additional steps can be performed with any specific aspect or combination of aspects of the disclosed methods.

Herein, the term “sublingual” means placing a drug or pharmaceutical under a user's tongue to dissolve and absorb same into a user's blood directly through the tissue there.

The invention provides a kit for migraine management, comprising a first container having a first therapeutically effective dosage of an efficacious pharmaceutical compound useful for treating a medical condition; a second container having a second therapeutically effective dosage of an efficacious pharmaceutical compound useful for treating a medical condition; a third container having a third therapeutically effective dosage of an efficacious pharmaceutical compound useful for treating a medical condition; and one or more printed instructions setting forth the safe and effective use of the contents of said kit.

The kit further comprises a computer application (i.e., an “app”) usable on a personal mobile device by a user. Examples of suitable personal mobile devices herein are a cellphone, laptop computer, personal computer, computer pad device from brands like APPLE©, DELL©, SAMSUNG©, and ANDROID© to name but a few.

In practice, The kit for migraine management tracks the occurrence, severity and length of a user's migraine headache. The app may also contain printed instructions viewable on said personal mobile device.

Herein, the first therapeutically effective dosage of an efficacious pharmaceutical compound is administered sub-lingually, i.e., under the tongue. The second therapeutically effective dosage of an efficacious pharmaceutical compound herein is administered orally for consumption through the user's esophagus and into the user's stomach. The kit for the third therapeutically effective dosage of an efficacious pharmaceutical compound useful for treating a medical condition is topically administered and is best provided in the form of a cream.

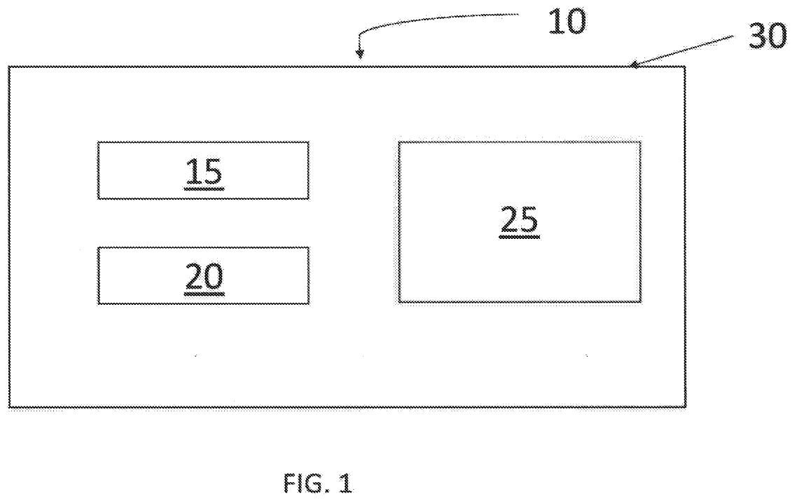

FIG. 1 provides a schematic drawing of the kit for migraine management 10 (or “kit 10”) herein. As shown, kit 10 comprises outer packaging 30 (i.e., “package 30”), first pills 15, second pills 20 and topical 25. First pills 15, second pills 20 and topical 25 are situated within package 30 in a manner that secures those items from incidental damage.

Package 30 may comprise materials well known by persons of skill in the art for use in packaging such as low density polyethylene (LDPE), high density polyethylene (HDPE), cardboard, paper and the like. Of course, mixtures thereof are also possible and well known.

First pills 15 and second pills 20 are preferably similarly housed within pharmaceutical grade pill bottles typically formed from HDPE that have a body and a cap configured to attach and lock to the body. Alternatively, first pills 15 and second pills 20 may be housed within LDPE packaging that forms a tearable pouch. Such pouch may, preferably, be resealable to maintain freshness of the contents therein, i.e., first pills 15 and second pills 20.

To provide the best relief possible for the symptoms above, the invention herein uses a propriety combination of ingredients as well as a unique delivery method to give users the best and fastest opportunity to experience relief and preferably prevention of migraine headaches. As noted hereinabove, the first part of the solution is a sublingual process using either a dissolvable tablet or a liquid with a dropper or even chewable gum. This process is designed to deliver ingredients as fast as possible to get the fastest relief, as well as minimize concern if vomiting accompanied. The desired ingredients are found in first pills 15. These ingredients have been proven to alleviant symptoms sustainably over placebo.

The ingredients in first pills 15 include caffeine (i.e., preferably a maximum dosage thereof); L-theanine (i.e., preferably a maximum dosage thereof); Meclizine HCL or known equivalent for anti-nausea benefits; ginger for taste and nausea remediation; magnesium for taste and pain alleviation; and preferably CBD oil where usable for enhanced delivery.

The second step herein includes either a single or dual pill delivery. Two pills are preferred for delivery to minimize size thereof especially for smaller child patients. Smaller pills enable ease of swallow at the onset of a headache if possible and potentially another either two or four hours later depending on how fast the ingredients of the pill(s) are able to be metabolized. The purpose of this pill is to reinforce the work of the sublingual dosage in some of the same ingredients as well as to deliver vitamins that have been proven to alleviate symptoms as well as shorten the downtime caused by migraines.

The ingredients in second pills 20 include an amount of magnesium (i.e., preferably a maximum dosage thereof), an amount of Arnica (i.e., preferably a maximum dosage thereof); vitamin B2 (riboflavin—i.e., preferably a maximum dosage thereof); California Poppy; an amount of Coenzyme Q10 (i.e., CoQ10) and preferably a maximum dosage thereof; lemon balm extract; another dosage of caffeine if preferred; yerba mate; ashwagandha, L-theanine, another dosage of ginger for its anti-nausea properties and alternatively another dosage of CBD in its oil form.

The third step provides ingredient delivery in the form of topical cream 25 which may be applied either by a gel applicator, cream in a tube or by a liquid spray. Delivery of cream 25 herein is designed to be applied to a user's forehead, temples, and/or neck at the onset of a migraine. It is used in conjunction with first pill 15, second pill(s) 20 and is absorbed into a user's skin. The ingredients herein have been proven to help alleviate symptoms of migraine headaches as well as providing a sensation of relief soon after topical application.

Ingredients for topical 25 include Magnesium; Lavender; Rosemary; Arnica; lemon balm extract; peppermint; spearmint; and ginger. A critical feature of topical 25 is that it should be both fast acting and quickly absorbing into a user's skin.

Persons of skill in the art will readily recognize that the listed ingredients for first pill 15, second pill 20 and topical 25 are provided singularly but also in combination with one-another. Persons of skill in the art that other safe, equivalent compounds of all the listed ingredients herein

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A kit for migraine management, comprising:
a. A first container having a first therapeutically effective dosage of an efficacious pharmaceutical compound useful for treating a medical condition;
b. A second container having a second therapeutically effective dosage of an efficacious pharmaceutical compound useful for treating a medical condition;
c. A third container having a third therapeutically effective dosage of an efficacious pharmaceutical compound useful for treating a medical condition; and
d. Printed instructions setting forth the safe and effective use of the contents of said kit.

2. The kit for migraine management of claim 1 wherein said kit further comprises a computer application usable on a personal mobile device by a user.

3. The kit for migraine management of claim 2 wherein said computer application tracks the occurrence, severity and length of said user's migraine headache.

4. The kit for migraine management of claim 3 wherein said computer application contains said printed instructions viewable on said personal mobile device.

5. The kit for migraine management of claim 1 wherein said first therapeutically effective dosage of an efficacious pharmaceutical compound is administered sub-lingually.

6. The kit for migraine management of claim 1 wherein said second therapeutically effective dosage of an efficacious pharmaceutical compound is administered orally for consumption through said user's esophagus.

7. The kit for migraine management of claim 1 wherein said third therapeutically effective dosage of an efficacious pharmaceutical compound useful for treating a medical condition is topically administered.

8. The kit for migraine management of claim 7 wherein said third therapeutically effective dosage of an efficacious pharmaceutical compound is administered in the form of a cream.
